(19) United States
(12) Patent Application Publication
(10) Pub. No.: US 2006/0024818 A1
(43) Pub. Date: Feb. 2, 2006

(54) Safety pouch for cryo-preservation of staminal cells or similar blood components

(76) Inventor: Mario Conconi, Monza (IT)

Correspondence Address: JACOBSON HOLMAN PLLC, 400 SEVENTH STREET N.W., SUITE 600, WASHINGTON, DC, 20004, US

(21) Appl. No.: 11/183,821

(22) Filed: Jul. 19, 2005

(30) Foreign Application Priority Data
Jul. 27, 2004 (IT) MI2004A001517

(51) Int. Cl. C12M 3/00

(52) U.S. Cl. 435/307.1; 604/409; 383/40

(57) ABSTRACT

A pouch for cryo-preservation of staminal cells or similar blood components is described, comprising a main pouch (2) containing the substance (4) to be preserved and relative means (5, 6) for the communication with the outside, characterised in that it comprises in addition an external perfectly sealed secondary pouch (3) containing said main pouch (2) and said communication means (5, 6).

The present invention concerns a safety pouch for cryo-preservation of staminal cells or similar blood components.

Current pouches for staminal cells, made of suitable plastic material (as for instance Ethyl Vinyl Acetate/E.V.A.) and opportunely sealed, are usually preserved at very low temperature (minimum reachable −196° C., usually −130° C. or −150° C.) in cryo-biological containers working with liquid nitrogen.

It can occur that the pouch is not perfectly watertight and therefore its content can get contaminated by the external atmosphere or can in turn contaminate the same, therefore with the risk of contaminating one of the other several pouches contained in the cryo-biological container.

Object of the present invention is to provide a device that is capable to perfectly isolate the pouch containing the staminal cells or other similar blood components, thus guaranteeing its perfect cryo-preservation.

According to the invention such object is attained with a pouch for cryo-preservation of staminal cells or similar blood components, comprising a main pouch, containing the substance to be preserved and relative means for the communication with the outside, and an external perfectly saleable secondary pouch, containing said main pouch and said communication means.

The presence of the external pouch allows to handle the main pouch in conditions of absolute tranquillity and safety, the substance contained therein being not contaminable and therefore ready to be administered to the patient.

These and other characteristics of the present invention will become evident from the following detailed description of an embodiment thereof that is illustrated as a non-limiting example in the enclosed drawings, in which:

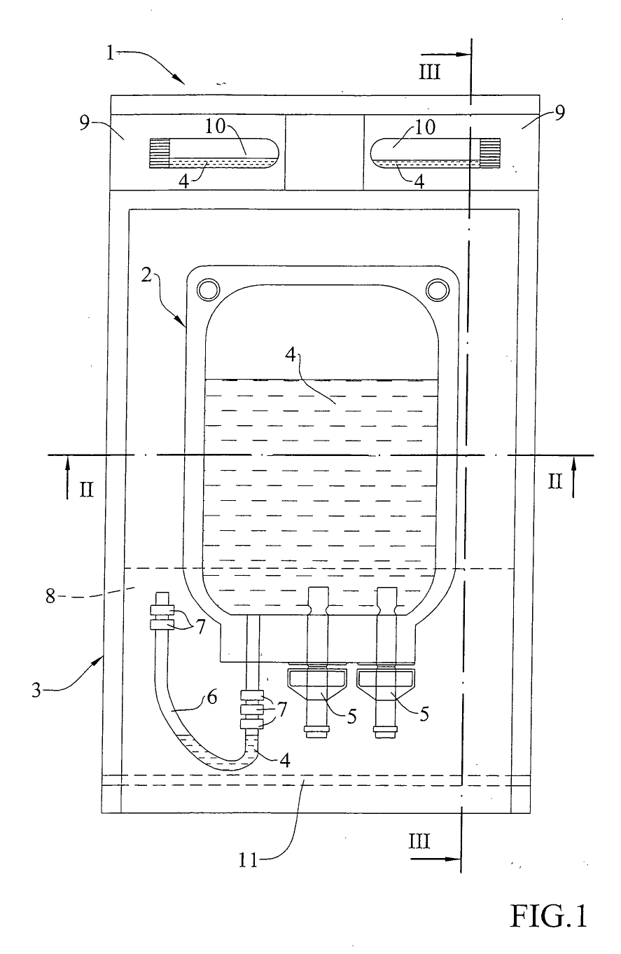

FIG. 1 shows a front view of a safety pouch according to the present invention;

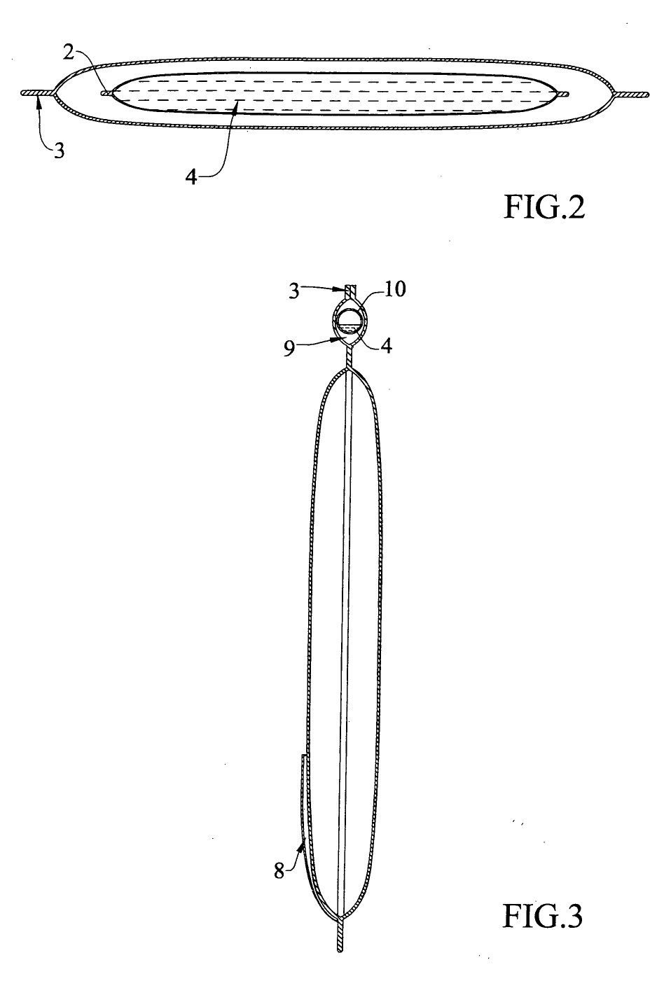

FIG. 2 shows a section view according to line II-II in FIG. 1;

FIG. 3 shows a section view according to line III-III in FIG. 1.

FIG. 1 shows a safety pouch 1 comprising an internal main pouch 2 and an external secondary pouch 3 containing said internal main pouch 2. The internal main pouch 2 contains a substance 4 to be preserved that can be introduced or extracted by means of a pair of frangible connectors 5 and a loading tube 6, both completely contained in the external secondary pouch 3.

Pinchings 7 guarantee the watertightness of the loading tube 6 once the internal main pouch 2 has been filled.

The external secondary pouch 3 comprises an external pocket 8 suitable to house tags with information for the identification of the substance 4, and a pair of housings 9 suitable to accommodate test-tubes 10 containing the same substance 4.

A seal 11 guarantees the perfect watertightness of the external secondary pouch 3.

The pouch 1 can be handled in total safety by the user. If there is the need to analyse the substance one can use the test-tubes 10 without having to open the external pouch 3.

Since the aforesaid analysis could not give absolute guarantee, because for a human error the test-tubes could be not representative of the substance 4, for greater safety before the introduction of the substance into the patient it is possible to use the substance contained in the loading tube 6, which can be extracted from the external pouch 3 by forcing the seal 11.

At the end of the loading of the internal main pouch 2 one proceeds in fact to the sealing of the loading tube 6 by means of the pinchings 7 which isolate a length of the tube 6 where a small amount of the substance remains which represents with absolute certainty the substance 4 contained in the internal main pouch 2, since it is a portion of the same.

Once the integrity of the substance 4 contained in the loading tube 6 has been verified one can proceed with the use of the same by breaking the frangible connectors 5 and by connecting them through appropriate infusion sets (not shown) with the patient.

1. Safety pouch for cryo-preservation of staminal cells or blood similar components, comprising a main pouch (2) containing the substance (4) to be preserved and relative means for the communication (5, 6) with the outside, characterised in that it comprises in addition an external perfectly sealable secondary pouch (3) containing said main pouch (2) and said communication means (5, 6).

2. Pouch according to claim 1, characterised in that said external secondary pouch (3) comprises at least one first external pocket (8) suitable to accommodate tags with information for the identification of the substance (4).

3. Pouch according to claim 1, characterised in that it includes at least one housing (9) for a testtube (10) containing the same substance (4) found in the main pouch (2).

4. Pouch according to claim 1, characterised in that said communication means (5, 6) comprise at least one frangible connector (5) and at least one loading tube (6).
